(19) United States
(12) Patent Application Publication
(10) Pub. No.: US 2022/0280334 A1
(43) Pub. Date: Sep. 8, 2022

(54) DRY-TYPE SEAT CUSHION CONFIGURED TO EMIT FAR INFRARED RAYS

(71) Applicant: RADIANCE CO., LTD., Seongnam (KR)

(72) Inventors: Se Yeong KIM, Seoul (KR); Dong Woo KIM, Seoul (KR)

(21) Appl. No.: 17/369,621

(22) Filed: Jul. 7, 2021

(30) Foreign Application Priority Data
Mar. 3, 2021 (KR) 10-2021-0027898

(51) Int. Cl. A61F 7/00; A47C 7/74

(52) U.S. Cl. CPC A61F 7/007; A47C 7/748; A61F 7/08

(57) ABSTRACT

Proposed is a dry-type seat cushion configured to emit far infrared rays, the seat cushion being capable of emitting far infrared rays toward a user's body through a hot wire type heating element that performs a heat generation operation and emits far infrared rays simultaneously, and is not only capable of being used freely regardless of time and place through a wired and wireless power supply method but is also able to maximize power consumption efficiency by applying a power saving function, which automatically controls the heat generation operation on the basis of the automatic human body sensing function.

### CROSS REFERENCE TO RELATED APPLICATION

The present application claims priority to Korean Patent Application No. 10-2021-0027898, filed Mar. 3, 2021, the entire contents of which is incorporated herein for all purposes by this reference.

### BACKGROUND OF THE INVENTION

### Field of the Invention

The present disclosure relates to a dry-type seat cushion configured to emit far infrared rays. More particularly, the present disclosure relates to a dry-type seat cushion configured to emit far infrared rays, the seat cushion being capable of emitting far infrared rays toward a user's body through a hot wire type heating element that performs a heat generation operation and emits far infrared rays simultaneously, and is not only capable of being used freely regardless of time and place through a wired and wireless power supply method but is also able to maximize power consumption efficiency by applying a power saving function, which automatically controls the heat generation operation on the basis of the automatic human body sensing function.

### Description of the Related Art

Recently, due to westernization of modern society and improvement of standards of living, time spent in a chair is gradually increasing in recent years. The design and structure of the chair are being developed; however, such a chair is aimed to reduce fatigue of a human body passively by being made to adapt to an original body structure and a posture of the human body. Accordingly, such a chair may not relieve fatigue transferred to and accumulated on body parts such as the perineum and the like including the anus that are directly in close contact with the chair for a long time, and an occurrence of various diseases or disorders such as hemorrhoids, loss of energy, urinary incontinence, prostate inflammatory disease, menstrual irregularity, and the like are increasing.

Therefore, in recent years, sitz bath products providing an effect corresponding to a sitz bath to a user by using a product equipped with a heat generation means such as a seat mat and a seat cushion have been developed and commercialized.

However, in the case of the conventional sitz bath products, the power supply is accomplished using a wired supply method, so the conventional sitz bath products have a problem of being limited in time and place because of incapable of being used in a place without an outlet.

In addition, a planar heating element of a large area is to be used to emit the far infrared rays, and in this case, the planar heating element is incapable of completely connecting the connection part connecting the electric wire and the hot wire (heating element) thereto. Therefore, there is a problem in that a poor connection and fire risk exist.

In addition, most of the conventional sitz bath products use an automatic temperature control method through a program, so the user may not freely adjust the temperature used, thereby having a problem and inconveniency in that detailed temperature control may not be possible.

The foregoing is intended merely to aid in the understanding of the background of the present disclosure and is not intended to mean that the present disclosure falls within the purview of the related art that is already known to those skilled in the art.

### SUMMARY OF THE INVENTION

Accordingly, the present disclosure has been made keeping in mind the above problems occurring in the related art, and the present disclosure is intended to provide a dry-type seat cushion configured to emit far infrared rays, the seat cushion being capable of emitting far infrared rays toward a user's body through a hot wire type heating element that performs a heat generation operation and emits far infrared rays simultaneously, and is not only capable of being used freely regardless of time and place through a wired and wireless power supply method but is also able to maximize power consumption efficiency by applying a power saving function, which automatically controls the heat generation operation on the basis of the automatic human body sensing function.

In order to achieve the above objective, according to one aspect of the present disclosure, there may be provided a dry-type seat cushion configured to emit far infrared rays, the seat cushion including: a cushion body unit 110 forming a sitting space for a user to sit and having a through-hole 110a formed in a center thereof in a height direction; a far infrared ray emitting unit 120 provided on the cushion body unit 110 and configured to emit the far infrared rays through heat generation; a power supply unit 130 configured to receive power by being connected to any one of an external battery or an external power supply and configured to apply the received power to the far infrared ray emitting unit 120; and a controller 140 configured to control an operation of the far infrared ray emitting unit 120.

In the embodiment, the far infrared ray emitting unit 120 may be positioned at a lower side of the cushion body unit 110, wherein the far infrared rays to be emitted through the far infrared ray emitting unit 120 may be emitted in an upward direction through the through-hole 110a formed in the cushion body unit 110.

In the embodiment, the power supply unit 130 may have one side electrically connected to the far infrared ray emitting unit 120 and an opposite side provided with a DC jack connection port 131 and may include a battery gender 130a having one side electrically connected to the DC jack connection port 131 and an opposite side configured to be electrically connected to an external auxiliary battery.

In the embodiment, the power supply unit 130 may have one side electrically connected to the far infrared ray emitting unit 120 and an opposite side provided with a DC jack connection port 131 and may include an AC adapter 130b having one side electrically connected to the DC jack connection port 131 and an opposite side configured to be electrically connected to an external AC power supply terminal.

In the embodiment, the cushion body unit 110 may further include: a weight sensing unit 150 provided therein and configured to generate a weight sensing signal by sensing a weight of the user sitting on the cushion body unit 110, wherein the controller 140 may be configured to automatically control an ON/OFF operation of the far infrared ray emitting unit 120 on the basis of the weight sensing signal.

In the embodiment, the seat cushion may further include: an illuminance sensing unit 160 positioned at the through-hole 110a and configured to sense a change in illuminance of the through-hole 110a when the through-hole 110a is shaded as the user sits on the cushion body unit 110, thereby generating an illuminance sensing signal, wherein the controller may be configured to automatically control an ON/OFF operation of the far infrared ray emitting unit 120 on the basis of the illuminance sensing signal.

In the embodiment, the seat cushion may further include: a temperature sensing unit 170 positioned at the through-hole 110a and configured to sense a change in temperature of the through-hole 110a in real time, thereby generating a temperature sensing signal, wherein the controller 140 may be configured to compare the temperature sensing signal with a preset temperature value and then to automatically control a heat generation state of the far infrared ray emitting unit 120 according to a temperature difference, thereby allowing the temperature of the through-hole 110a to be maintained in a state of the preset temperature value.

In the embodiment, the controller 140 may include: a control means 140a electrically connected to the controller 140 and configured to generate an operation signal through a user operation and then to transfer the operation signal to the controller 140, wherein the control means 140a may include: a power control switch 140a-1 configured to physically control an ON/OFF operation of the far infrared ray emitting unit 120; and a dial switch 140a-2 configured to physically control heat generation temperature of the far infrared ray emitting unit 120 through a user operation.

In the embodiment, the control means 140a may further include: a mode control switch 140a-3 configured to automatically control an operation mode of the far infrared ray emitting unit 120 at the controller 140.

As described above, according to the present disclosure, there is an advantage in that a dry-type seat cushion configured to emit far infrared rays can be freely used regardless of time and place through a wired and wireless power supply method and, in particular, can emit the far infrared rays toward a user's body through a hot wire type heating element that performs a heat generation operation and emits far infrared rays simultaneously.

In particular, according to the present disclosure, there is an advantage of being capable of maximizing convenience and extending a range of use by enabling the dry-type seat cushion configured to emit far infrared rays to be used by using an auxiliary battery for a smartphone that anyone carries.

In addition, according to the present disclosure, there is an advantage of maximizing power consumption efficiency by applying a power saving function that automatically controls heat generation operation on the basis of an automatic human body sensing function.

In particular, according to the present disclosure, both a battery gender configured to connect the auxiliary battery and an adapter configured to supply AC power are provided, thereby having an advantage of compatibility capable of supplying power supply by connecting an adapter having the same output as the auxiliary battery when it is difficult to use the auxiliary battery.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view showing a configuration of a dry-type seat cushion 100 configured to emit far infrared rays according to an embodiment of the present disclosure;

FIG. 2 is a view showing an overall shape of the dry-type seat cushion 100 configured to emit the far infrared rays shown in FIG. 1;

FIGS. 3A to 3C are views showing a power supply unit 130 shown in FIG. 2 in more detail; and

FIG. 4 is a view showing a control means 140a shown in FIG. 2 in more detail.

### DETAILED DESCRIPTION OF THE INVENTION

Hereinbelow, with reference to the accompanying drawings, embodiments of the present disclosure will be described in detail so as to be easily implemented by those of ordinary skill in the art to which the present disclosure pertains. The present disclosure may be embodied in many different forms and is not limited to the embodiments described herein.

In order to clearly describe the present disclosure, parts irrelevant to the description are omitted, and the same reference numerals are assigned to the same or similar elements throughout the specification.

In addition, in various embodiments, components having the same configuration will be described only in the representative embodiment using the same reference numerals, and in other embodiments, only configurations different from the representative embodiment will be described.

Throughout the specification, when a part is “connected” with another part, it includes not only the case where the part is “directly connected” but also the case where the part is “indirectly connected” with another member interposed therebetween. In addition, when a part “includes” a certain component, the part may mean further including other components rather than excluding other components unless particularly otherwise stated.

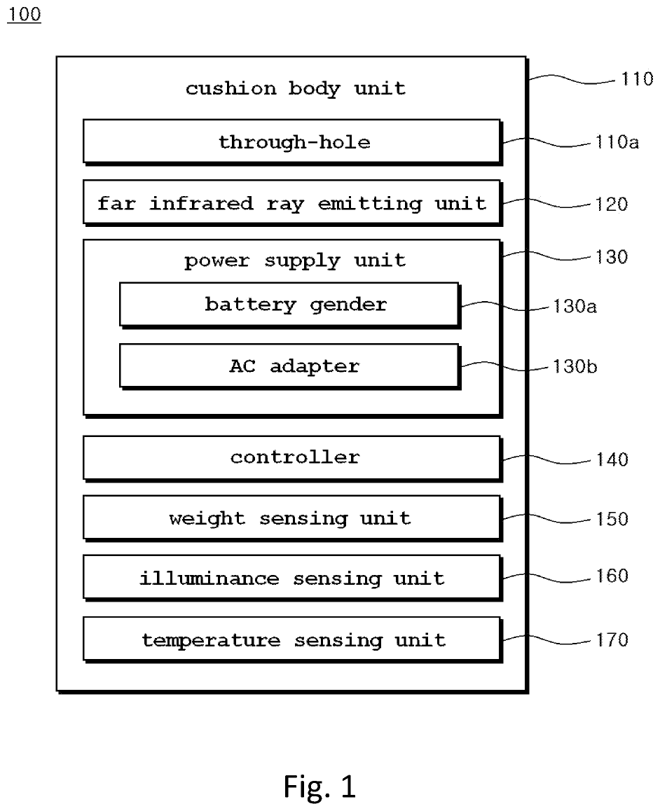

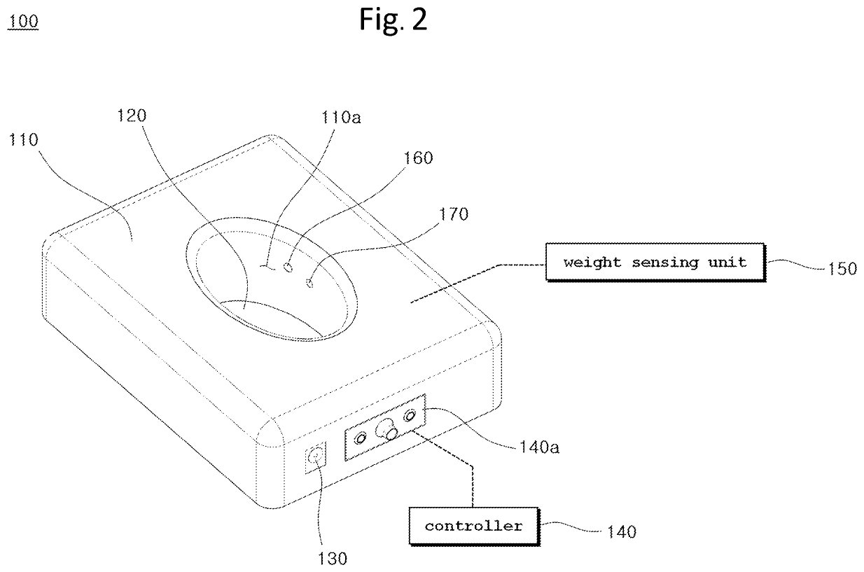

FIG. 1 is a view showing a configuration of a dry-type seat cushion 100 configured to emit far infrared rays according to an embodiment of the present disclosure, and FIG. 2 is a view showing an overall shape of the dry-type seat cushion 100 configured to emit the far infrared rays shown in FIG. 1.

Looking at FIGS. 1 and 2 together, the dry-type seat cushion 100 configured to emit the far infrared rays according to the embodiment of the present disclosure may be configured to largely include a cushion body unit 110, a far infrared ray emitting unit 120, a power supply unit 130, a controller 140, a weight sensing unit 150, an illuminance sensing unit 160, and a temperature sensing unit 170.

First, the cushion body unit 110 is configured to include a settling space (or sitting space) formed for a user to settle, a shock absorber filled therein for shock absorption and alleviation, and a through-hole 110a formed in a height direction in the center.

At this time, the through-hole 110a is formed to have a bottom surface not being perforated and an upper part only being perforated, and the far infrared ray emitting part 120 is provided on the bottom surface. Here, the size and position of the through-hole 110a are determined from an entire surface of the cushion body unit 110 so as to include the periphery of both the anus and the genital area of a man or woman when a user sits. At this time, the through-hole 110a may be determined to position in an exact center or to a position close to the center with an upper side surface of the cushion body unit 110 as a reference. In addition, the size of the through-hole 110a may be formed in an elliptical shape, wherein the lengths in a long direction and a short direction of the ellipse may be 10 cm to 28 cm and 5 cm to 18 cm, respectively.

The far infrared ray emitting part 120 is provided on a lower side of the cushion body unit 110 and is configured to serve to emit the far infrared rays through heat generation.

Here, in the case of the far infrared ray emitting unit 120, a plurality of ultra-fine wire strands, emitting the far infrared rays while performing heat generation due to a resistance value when a current flows therethrough, is combined to be in contact with each other, and then is formed into one bundle, thereby making a single strand hot wire. At this time, in order to make the bundles into an effective geometrical structure in which electric dipole radiation emitting the far infrared rays may be well radiated, the bundles are grouped into at least two having resistance values different from one another, and then one ultra-fine wire strand or at least two ultra-fine wire strands are made to have an equal resistance value by each group, wherein the groups have resistance values different from one another. In general, a popularized smartphone auxiliary battery has an output voltage of 5V, and the maximum discharge current is mostly 2A. When the maximum discharge current exceeds 2A, an auxiliary battery protection circuit is activated, whereby the discharge power is automatically shut off.

Therefore, while the popularized 5V auxiliary battery is applied to perform heat generation operation but, in order to prevent the auxiliary battery protection circuit from operating, a total resistance value of the hot wires (heating elements) used in the far infrared ray emitting unit 120 is formed with 2.5 ohms to 3.5 ohms in the present disclosure.

In the case of such a hot wire, ultra-fine wires of a plurality of strands are made using two different materials (alloy metal), and each ultra-fine wire strand is combined to make one bundle that is the hot wire. At this time, the ultra-fine wire of type 1 material is NASLON, a steel fiber, wherein the thickness of one ultra-fine wire strand is to become 12 nanometers and the number of strands is to become 550.

An ultra-fine wire of another type 1 material is a single metal of nickel and copper, wherein the mixing ratio of the single metal is made to become 20 to 25 weight percent of nickel and 75 to 80 weight percent of copper, the thickness of one ultra-fine wire strand becomes 100 nanometers (at this time, the resistance value of one strand is to become 36 ohms), and the number of the strands is to become 14.

A total of 564 ultra-fine wire strands are allowed to be brought into contact with one another in a single bundle so as to be electrically connected, wherein a primary coating is formed by wrapping an outer surface of the synthesized ultra-fine wire bundle (strands) with high-temperature fibers to be overlapped, and a secondary coating is formed thereon again with Teflon material.

At this time, at least one of aramid, polyarylate, and Zylon may be applied as a high-temperature fiber.

In particular, the hot wire, made in this way to have a resistance value of 2.3 ohms per 1 m. In this case, the hot wire is formed to have the length of 1.09 m to 1.52 m so that the resistance value is 2.5 ohms to 3.5 ohms, thereby being used as the hot wire (heating element) of the far infrared ray emitting unit 120 of the present disclosure.

Such a far infrared ray emitting unit 120 emits the far infrared rays emitted through the heating element to the body of the user sitting on the cushion body unit 110 through the through-hole 110a formed in the cushion body unit 110.

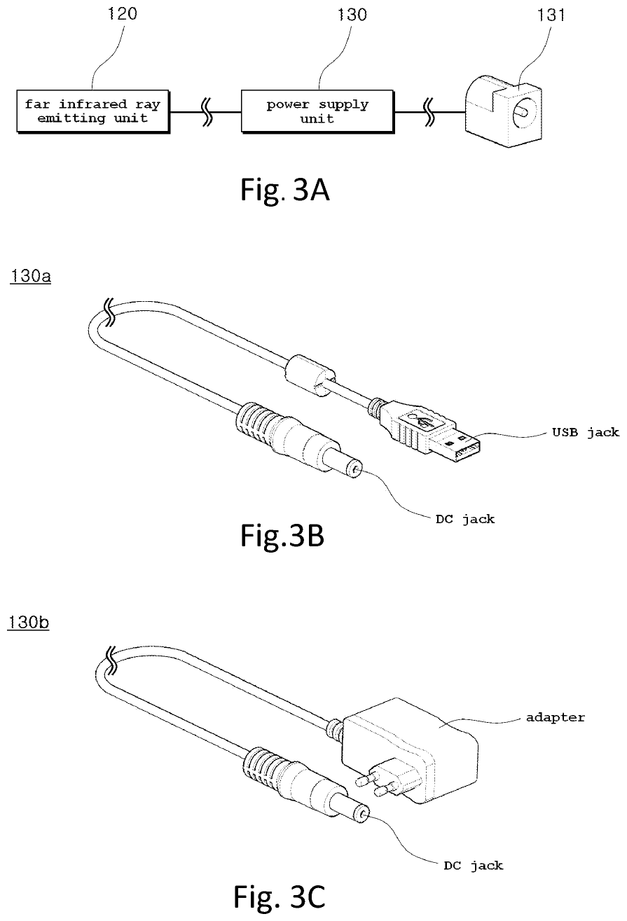

The power supply unit 130 is connected to any one of an external battery (smartphone auxiliary battery and the like) or an external power supply (AC power supply) to receive power and serves to apply the received power to the far infrared ray emitting unit 120. This will be described in more detail with reference to FIGS. 3A to 3C.

FIGS. 3A to 3C are views showing the power supply unit 130 shown in FIG. 2 in more detail.

Looking at FIG. 3A, the power supply unit 130 is configured to have basically one side electrically connected to the far infrared ray emitting unit 120 and an opposite side provided with a DC jack connection port 131, wherein the DC jack connection port 131 is configured to be connected to a battery gender (130a) configured to be electrically connected to an external auxiliary battery or to an AC adapter 130b configured to be electrically connected to an external AC power.

The battery gender 130a has one side provided with a DC jack configured to be connected to the DC jack connection port 131 and an opposite side provided with a USB jack configured to be connected to the external auxiliary battery. In addition, the AC adapter 130b has one side provided with a DC jack configured to be connected to the DC jack connection port 131 and an opposite side provided with an adapter configured to be connected to the external AC power supply terminal.

At this time, the DC jack of the battery gender 130a and the DC jack of the AC adapter 130b may be freely detachably coupled to the DC jack connection port 131, respectively, so the battery gender 130a or the AC adapter 130b may be interchangeably connected freely to the DC jack connection port 131 depending on the power supply environment.

For example, when infrared ray emitting unit 120 needs to be applied with power using the external auxiliary battery, the DC jack of the battery gender 130a as shown in FIG. 3B may be inserted into the DC jack connection port 131 so as to be combined therewith, and when the infrared ray emitting unit 120 needs to be applied with power using the AC power supply, the DC jack of the AC adapter 130b as shown in FIG. 3C may be inserted into the DC jack connection port 131 so as to be combined therewith.

Returning back to FIGS. 1 and 2 again, in a process in which the power supplied from the external battery or the external AC power terminal connected to the power supply unit 130 is applied to the far infrared ray emitting unit 120, the controller 140 serves to automatically control an application state of the applied power and an operation of the far infrared ray emitting unit 120 accordingly. Here, such a controller 140 may imply a kind of MCU.

More specifically, the far infrared ray emitting unit 120 is generally designed such that a power supply state automatically becomes an ON state when the power is supplied, but in the present disclosure, the controller 140 becomes to control the ON state or the OFF state of the far infrared ray emitting unit 120 midway.

That is, even when the power supplied through the power supply unit 130 is applied to the far infrared ray emitting unit 120, only when the controller 140 changes the operation state of the far infrared ray emitting unit 120 to the ON state, the far infrared ray emitting unit 120 becomes to be the ON state, thereby being activated.

Conversely, even when the power is applied to the far infrared ray emitting unit 120 through the power supply 130, when the controller 140 changes the operation state of the far infrared ray emitting unit 120 to the OFF state, the far infrared ray emitting unit 120 becomes to be the OFF state and stops the operation thereof.

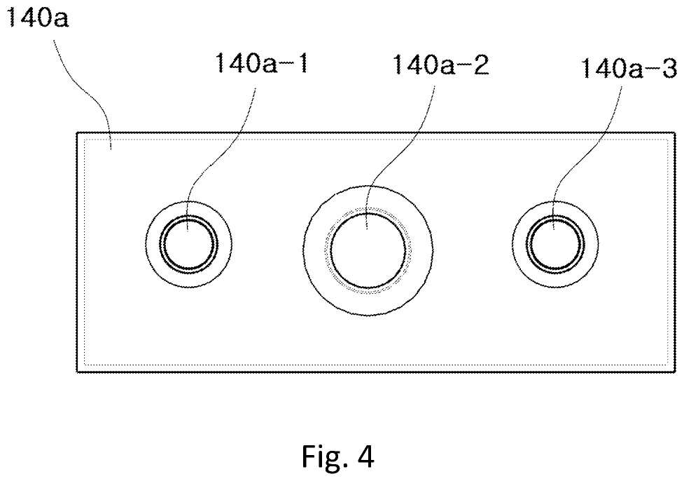

Such a controller 140 is electrically connected to the control means 140a provided on one side of the cushion body unit 110, which will be described in more detail with reference to FIG. 4.

FIG. 4 is a view showing a control means 140a shown in FIG. 2 in more detail.

Looking at FIG. 4, the control means 140a may largely include a power control switch 140a-1 configured to physically control an ON/OFF operation of the far infrared ray emitting unit 120 through a user operation, and a dial switch 140a-2 configured to physically control the heat generation temperature of the far infrared ray emitting unit 120 through a user operation. In addition, in an additional embodiment, the control means 140a may be configured to further include a mode control switch 140a-3 configured to physically control an operation mode of the far infrared ray emitting unit 120 through a user operation.

More specifically, the power control switch 140a-1 refers to a kind of toggle switch configured to physically change the ON or OFF state of the far infrared ray emitting unit 120 through the user operation.

Therefore, when the power control switch 140a-1 is operated in the ON state through the user operation, the operation state of the far infrared ray emitting unit 120 is changed to the ON state, and heat generation and far infrared ray emission are started. Conversely, when the power control switch 140a-1 is operated to the OFF state through user operation, the operation state of the far infrared ray emitting unit 120 is changed to the OFF state, and the heat generation and far infrared ray emission are stopped. Such a power control switch 140a-1 implies an immediate operation control reflecting the user operation regardless of the automatic control of the controller 140.

The dial switch 140a-2 refers to a kind of variable resistance switch configured to physically change the heat generation temperature through the far infrared ray emitting unit 120 in detail through a user operation. Accordingly, as the dial switch 140a-2 is rotated clockwise or counterclockwise through the user operation, the heat generation temperature of the far infrared ray emitting unit 120 may be very finely adjusted. In this case, the extent to which the heat generation temperature is able to be adjusted through the dial switch 140a-2 is not limited.

On the other hand, the dry-type seat cushion 100 configured to emit the far infrared rays according to the present disclosure is designed so as to perform heat generation and the far infrared ray emission even through the supply power of the external auxiliary battery, so it is necessary to minimize unnecessary power consumption.

Accordingly, the controller 140 of the dry-type seat cushion 100 configured to emit far infrared rays according to the present disclosure becomes to automatically control the heat generation operation of the far infrared ray emitting unit 120 by itself even without the user operation.

To this end, the controller 140 of the present disclosure is connected to the weight sensing unit 150, the illuminance sensing unit 160, and the temperature sensing unit 170 and configured to receive a sensing signal from each unit, thereby controlling the heat generation operation of the far infrared ray emitting unit 120 automatically. At this time, the controller 140 may include a mode control element (switching element) such as an FET that is automatically operated by a program. Accordingly, by automatically manipulating the FET programmatically on the basis of a real-time sensing signal sensed through the weight sensing unit 150, the illuminance sensing unit 160, and the temperature sensing unit 170, the controller 140 may automatically control the heat generation state of the far infrared ray emitting unit 120.

More specifically, the weight sensing unit 150 is provided at a position, where the user sits, of the cushion body unit 110 and, after sensing a change in weight according to the state when the user sits on or stands up from the seat, generates a weight sensing signal to transfer to the controller 140.

In this case, the weight sensing unit 150 may be, for example, applied with a pressure sensor using a piezoelectric element, thereby sensing a change in the weight when the user sits on the cushion body unit 110.

The controller 140 recognizes a state when the user sits on the basis of the weight sensing signal. When it is determined that the user sits, the operation of the far infrared ray emitting unit 120 is automatically changed to the ON state so that heat generation and far infrared ray emission are started, and on the contrary, when it is determined that the user stands up from the seat, the operation of the far infrared ray emitting unit 120 is automatically changed to the OFF state so that the heat generation and far infrared ray emission are stopped.

The illuminance sensing unit 160 is positioned on an inner surface of the through-hole 110a of the cushion body unit 110 and, as the user sits on the cushion body unit 110, after sensing a change in illuminance around the through-hole 110a, generates an illuminance sensing signal to transfer to the controller 140.

At this time, the illuminance sensing unit 160 may be applied with an optical sensor (illuminance sensor) and, when the user sits on the cushion body unit 110, sense a situation in which the through-hole 110a is darkened by the user's body. That is, the illuminance sensing unit 160 determines the moment when the light entering the through-hole 110a is blocked and generates an illuminance sensing signal on the basis thereof.

The controller 140 recognizes a state when the user sits on the basis of the illuminance sensing signal. When it is determined that the user sits (when the illuminance value falls to a point no higher than a preset illuminance value), the controller 140 automatically changes the operation of the far infrared ray emitting unit 120 to the ON state, thereby allowing the heat generation and the far infrared ray emission to be started. Conversely, when it is determined that the user stands up from the seat (when the illuminance value recovers or rises to a point no lower than the preset illuminance value), the controller 140 automatically changes the operation of the far infrared ray emitting unit 120 to the OFF state, thereby allowing the heat generation and far infrared ray emission to be stopped.

The temperature sensing unit 170 is positioned on the inner surface of the through hole 110a of the cushion body unit 110, and generates a temperature sensing signal by sensing the heat generation temperature of the far infrared ray emitting unit 120 positioned at a side therebelow in real time, thereby transferring the generated temperature sensing signal to the controller 140.

In this case, the controller 140 may set a reference temperature for automatic temperature control through the aforementioned control means 140a. Therefore, the controller 140 automatically recognizes whether the temperature sensing signal transferred through the temperature sensing unit 170 has fallen from or increased to the preset reference temperature and, then when it is determined that the temperature falls to a point lower than the preset reference temperature, raises the heat generation temperature of the far infrared ray emitting unit 120 to be the preset reference temperature. Conversely, when it is determined that the temperature is higher than the reference temperature, the heat generation temperature of the far infrared ray emitting unit 120 is lowered to the preset reference temperature. By automatically repeating this process, the user may always have a constant feeling of warmth.

On the other hand, the control means 140a of the present disclosure may be further provided with a mode control switch 140a-3 configured to start an independent automatic control operation of the controller 140 through the user operation.

Unlike the power control switch 140a-1 and the dial switch 140a-2 described above, the mode control switch 140a-3 is a switch for a kind of power saving mode, and under such a mode, the operation state and heat generation state of the far infrared ray emitting unit 120 is controlled not by the user directly but by the controller 140 automatically. The controller 140 described above may automatically control the heat generation state of the far infrared ray emitting unit 120 after receiving real-time sensing signals from the weight sensing unit 150, the illuminance sensing unit 160, and the temperature sensing unit 170 and, when the mode control switch 140a-3 is selected by the user operation, the automatic control operation through the controller 140 described above may also be immediately executed.

Although the above has been described with reference to the exemplary embodiments of the present disclosure, it will be understood that those skilled in the art may variously modify and change the present disclosure without departing from the spirit and scope of the present disclosure as set forth in the claims below.

What is claimed is:

1. A dry-type seat cushion configured to emit far infrared rays, the seat cushion comprising:
a cushion body unit (110) forming a sitting space for a user to sit and having a through-hole (110a) formed in a center thereof in a height direction;
a far infrared ray emitting unit (120) provided on the cushion body unit (110) and configured to emit the far infrared rays through heat generation;
a power supply unit (130) configured to receive power by being connected to any one of an external battery or an external power supply and configured to apply the received power to the far infrared ray emitting unit (120); and
a controller (140) configured to control an operation of the far infrared ray emitting unit (120).

2. The seat cushion of claim 1, wherein the far infrared ray emitting unit (120) is positioned at a lower side of the cushion body unit (110), wherein the far infrared rays to be emitted through the far infrared ray emitting unit (120) are emitted in an upward direction through the through-hole (110a) formed in the cushion body unit (110).

3. The seat cushion of claim 1, wherein the power supply unit (130) has one side electrically connected to the far infrared ray emitting unit (120) and an opposite side provided with a DC jack connection port (131) and includes a battery gender (130a) having one side electrically connected to the DC jack connection port (131) and an opposite side configured to be electrically connected to an external auxiliary battery.

4. The seat cushion of claim 1, wherein the power supply unit (130) has one side electrically connected to the far infrared ray emitting unit (120) and an opposite side provided with a DC jack connection port (131) and includes an AC adapter (130b) having one side electrically connected to the DC jack connection port (131) and an opposite side configured to be electrically connected to an external AC power supply terminal.

5. The seat cushion of claim 1, wherein the cushion body unit (110) further comprises:
a weight sensing unit (150) provided therein and configured to generate a weight sensing signal by sensing a weight of the user sitting on the cushion body unit (110),
wherein the controller (140) is configured to automatically control an ON/OFF operation of the far infrared ray emitting unit (120) on the basis of the weight sensing signal.

6. The seat cushion of claim 1, further comprising:
an illuminance sensing unit (160) positioned at the through-hole (110a) and configured to sense a change in illuminance of the through-hole (110a) when the through-hole (110a) is shaded as the user sits on the cushion body unit (110), thereby generating an illuminance sensing signal,
wherein the controller is configured to automatically control an ON/OFF operation of the far infrared ray emitting unit (120) on the basis of the illuminance sensing signal.

7. The seat cushion of claim 1, further comprising:
a temperature sensing unit (170) positioned at the through-hole (110a) and configured to sense a change in temperature of the through-hole (110a) in real time, thereby generating a temperature sensing signal,
wherein the controller (140) is configured to compare the temperature sensing signal with a preset temperature value and then to automatically control a heat generation state of the far infrared ray emitting unit (120) according to a temperature difference, thereby allowing the temperature of the through-hole (110a) to be maintained in a state of the preset temperature value.

8. The seat cushion of claim 1, wherein the controller (140) comprises:
a control means (140a) electrically connected to the controller (140) and configured to generate an operation signal through a user operation and then to transfer the operation signal to the controller (140),
wherein the control means (140a) comprises:
a power control switch (140a-1) configured to physically control an ON/OFF operation of the far infrared ray emitting unit (120); and
a dial switch (140a-2) configured to physically control heat generation temperature of the far infrared ray emitting unit (120) through a user operation.

9. The seat cushion of claim 8, wherein the control means (140a) further comprises:
a mode control switch (140a-3) configured to automatically control an operation mode of the far infrared ray emitting unit (120) at the controller (140).
